(19) United States
(12) Patent Application Publication
(10) Pub. No.: US 2023/0368879 A1
(43) Pub. Date: Nov. 16, 2023

(54) HEALTH AND MEDICAL HISTORY VISUALIZATION AND PREDICTION USING MACHINE-LEARNING AND ARTIFICIAL INTELLIGENCE MODELS

(71) Applicant: Cipherome, Inc., San Jose, CA (US)

(72) Inventors: Min Sang Kim, Sunnyvale, CA (US); Samuel Kim, La Palma, CA (US); Mijeong Ban, San Jose, CA (US); In Gu Lee, San Jose, CA (US)

(21) Appl. No.: 18/316,130

(22) Filed: May 11, 2023

Related U.S. Application Data
(60) Provisional application No. 63/341,942, filed on May 13, 2022.

(51) Int. Cl. G16H 15/00; G16H 10/60; G16H 40/63; G16H 50/70; G16H 50/30; G16H 20/10

(52) U.S. Cl. CPC G16H 15/00; G16H 10/60; G16H 40/63; G16H 50/70; G16H 50/30; G16H 20/10

(57) ABSTRACT

A health technology system provides various applications for providing intelligent informatics on health and medical histories of subjects. In one embodiment, the health technology system includes a visualization system for providing an interactive user interface (UI) for displaying health and medical history for a subject. Specifically, the visualization system obtains health and medical information for a subject and generates various elements on the UI for visualizing the health and medical history of the subject.

### CROSS-REFERENCE TO RELATED APPLICATIONS

This application claims the benefit of U.S. Provisional Application No. 63/341,942, filed on May 13, 2022, which is incorporated herein in its entirety.

### TECHNICAL FIELD

This disclosure relates generally to health technology applications, and particularly to visualization and analysis of electronic health and medical records using machine-learning.

### BACKGROUND

A health record system may store records related to health and medical history of a subject (e.g., patient in hospital or subject in clinical trial). In conventional health record systems, the health and medical history of the subject may include various events that are aggregated across different sources. For example, the records for a subject may include admission and visit records to a hospital, specialized medical procedures performed at medical facilities, test results from laboratories, prescriptions received from pharmacies, and the like. Moreover, each source may store the records in different data schemas, formats, and file spaces. Thus, it is difficult for a health provider (e.g., physician) or the subject to quickly identify and view relevant subsets of events for the subject.

### SUMMARY

A health technology system provides various applications for providing intelligent informatics on health and medical histories of subjects. In one embodiment, the health technology system includes a visualization system for providing an interactive user interface (UI) for displaying health and medical history for a subject. Specifically, the visualization system obtains health and medical information for a subject and generates various elements on the UI for visualizing the health and medical history of the subject.

In one embodiment, the health technology system includes a learning system that trains one or more machine-learning models and performs various inference and generative tasks using the trained machine-learning models. For example, a machine-learning model may be configured to receive a set of health and medical events outlined in the records of a subject and generate a prediction on whether a drug will generate an adverse drug reaction (ADR) when consumed by the subject.

In one embodiment, the visualization system, in conjunction with the learning system, also obtain predictions for the subject that are generated using artificial intelligence or machine-learning applications based on the health and medical records for the subject. The predictions can be used to generate potential treatment plans, flag medical procedures or prescriptions, generate likelihoods of future health and medical events for the subject that can be used by a health provider when treating the subject. The predictions can be displayed in an interactive and convenient manner on the UI.

### BRIEF DESCRIPTION OF DRAWINGS

FIG. 1 illustrates a system environment for a health technology system, according to one embodiment.

FIG. 2 illustrates a block architecture of the health technology system, according to one embodiment.

FIG. 3A illustrates a main interface generated by the visualization system, according to one embodiment.

FIG. 3B illustrates a continuation of the main interface generated by the visualization system, according to one embodiment.

FIG. 4 illustrates an example risk estimator for ASCVD, according to one embodiment.

FIG. 5 illustrates a main interface generated by the visualization system, according to another embodiment.

FIG. 6 illustrates a dialog box for simulating a new event for one or more subjects, according to one embodiment.

FIG. 7 illustrates a method of using machine-learning models trained using longitudinal medical records from a large population and obtaining the results by the visualization system, according to an embodiment.

FIG. 8 is an example flowchart for displaying health and medical records for a subject, according to one embodiment.

The figures depict various embodiments of the present disclosure for purposes of illustration only. One skilled in the art will readily recognize from the following discussion that alternative embodiments of the structures and methods illustrated herein may be employed without departing from the principles of the disclosure described herein.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

Disclosed is a configuration (including a system, a process, as well as a non-transitory computer readable storage medium storing program code) for generating a dynamic data conversion module and deploying the dynamic data conversion module on one or more client nodes in a learning environment.

### Overview

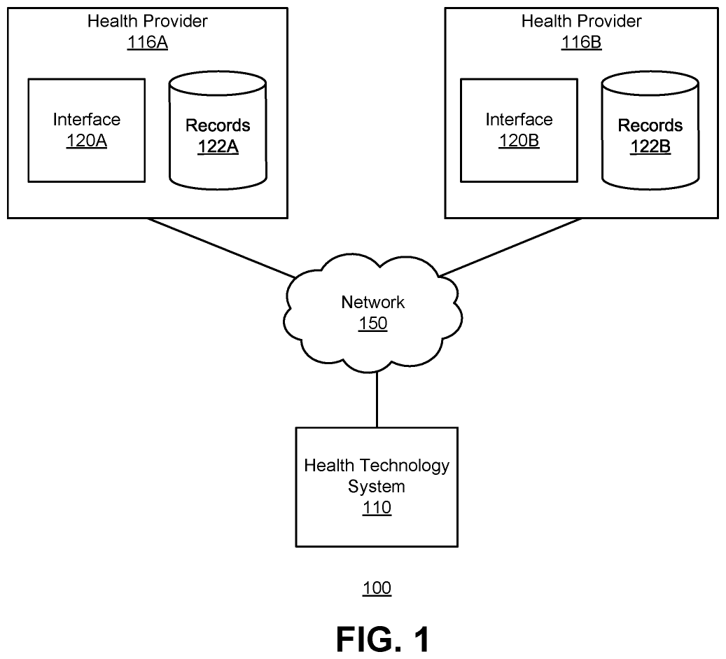

FIG. 1 illustrates a system environment 100 for a health technology system, according to one embodiment. The system environment 100 shown in FIG. 1 comprises a health technology system 110, one or more health providers 116A, 116B, and a network 150. In alternative configurations, different and/or additional components may be included in the system environment 100 and embodiments are not limited hereto.

The health technology system 110 provides various applications for providing intelligent informatics on health and medical histories of subjects (e.g., patients in a hospital, subjects in a clinical trial). In one embodiment, the health technology system 110 includes a repository of health records related to the health and medical history of a subject. In particular, in some existing health record systems, the health and medical history of the subject may include various events that are aggregated across different sources. For example, the records for a subject may include admission and visit records to a hospital, specialized medical procedures performed at medical facilities, test results from laboratories, prescriptions received from pharmacies, and the like. Moreover, each source may store the records in different data schemas, formats, and file spaces. Thus, it is difficult for a health provider (e.g., physician) or the subject to quickly identify and view relevant subsets of events for the subject.

Thus, in one embodiment, the health technology system 110 includes a visualization system that generates an interactive user interface (UI) for displaying health and medical history for a subject. Specifically, the visualization system obtains health and medical information for a subject and generates various elements on the UI for visualizing the health and medical history of the subject in an intelligent manner. In one embodiment, the visualization system obtains health and medical history for a subject that includes a plurality of health-related events and assigns the events to respective input categories. Specifically, an input category refers to a type of health or medical event, such as prescription events of a particular drug, medical and surgical procedures on a particular part of the body, adverse drug reactions (ADRs), hospital visits, and the like. For each input category, the visualization system may display a timeline of events pertaining to that input category.

The health technology system 110 may deploy one or more applications that users can use to access the various services of the health technology system 110. For example, the application may be a web application a user can access through a browser, a local application, such as a mobile application or an application native to the operating system of a computing system for a health provider 116. The health technology system 110 through the application on the computing system generates an interface to display the visualization. By presenting timelines of events for different input categories, the user of the client device can easily and quickly visualize subsets of events for each input category. The visualization system can help identify medical causes, deviations from standard protocols or treatment plans, likelihood of future medical events, and can provide a wholistic view of the medical history of the subject compared to existing systems.

In one embodiment, the health technology system 110 additionally includes a learning system that trains one or more machine-learning models and performs various inference and generative tasks using the trained machine-learning models. For example, a machine-learning model may be configured to receive a set of health and medical events outlined in the records of a subject and generate a prediction on whether a drug will generate an adverse drug reaction (ADR) when consumed by the subject.

In one embodiment, the health technology system 110 additionally includes an analytics system that provides information on potential issues, standard protocols and deviations from these standard protocols, recommendations, and the like, based on the health and medical history of a subject. In one instance, the analytics system is connected to one or more risk estimators that are, for example, widely recognized by the health and medical industry, such as the Atherosclerotic Cardiovascular Disease (ASCVD) Risk Estimator Plus from the American College of Cardiology (ACC).

In one embodiment, the visualization system of the health technology system 110, in conjunction with the learning system, also obtain predictions for the subject that are generated using artificial intelligence or machine-learning applications based on the health and medical records for the subject. The predictions can be used to generate potential treatment plans, flag medical procedures or prescriptions, generate likelihoods of future health and medical events for the subject that can be used by a health provider when treating the subject. The predictions can be displayed in an interactive and convenient manner on the interface. In one embodiment, the visualization system is further configured to highlight a particular region on the timeline (via color coding or hash) to indicate the likelihoods of one or more future events occurring for the subject based on events that are displayed on the interface.

The health providers 116 may each correspond to a health or medical service provider that provides one or more services to a subject. While the system environment 100 of FIG. 1 illustrates two health providers 116A and 116B, in practice, there may be many different types of health providers 116 that are in communication with the health technology system 110. For example, the health provider 116 may be a department of a hospital that provides various types of medical services, a pharmacy that fulfills drug prescriptions, a radiology clinic that provides diagnoses based on radiology images, a laboratory that runs one or more health-related tests for a subject, and the like.

The health provider 116 may include a computing system (e.g., local server) or control a remote computing system (e.g., cloud servers) that manages health-related data for the respective institution and/or organization. Thus, each health provider 116 may include a records datastore 122 storing records related to the health and medical history of one or more subjects. For example, health provider 116A as a department at a hospital may store visitation and diagnoses records for patients in a records datastore 122A. As another example, health provider 116B as a pharmacy may store prescription records for subjects in the records datastore 122B. A health provider 116 may store the records locally (e.g., on a local server, a private cloud, a disk on the health provider system) or may be stored on a cloud platform in a remote object datastore. A health provider 116 may store records according to a particular data schema that differs from schema of other health providers 116.

In one embodiment, the computing system for a health provider 116 further includes an interface 120 that allows the health provider 116 to interact with the services of the health technology system 110. For example, a health provider 116 may access an application (e.g., web application, mobile application, desktop application) of the health technology system 110 through interface 120. For example, health provider 116A may install a desktop application of the health technology system 110 and communicate via an interface 120A provided by the desktop application. As another example, health provider 116A may access a web application through an interface 120B generated by a browser.

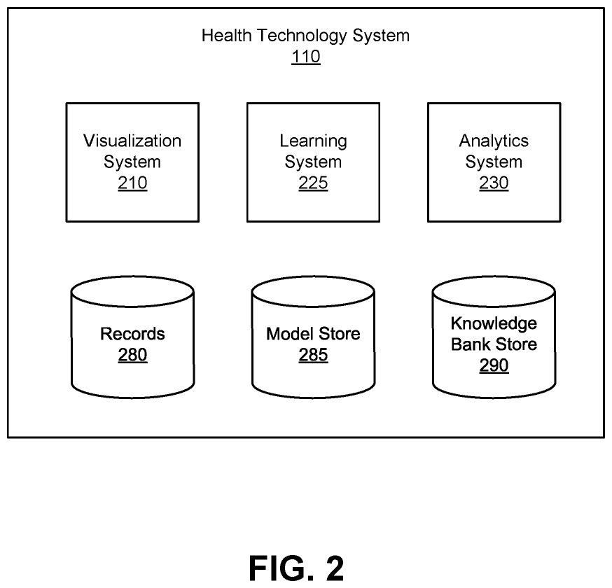

In one embodiment, the one or more health providers 116 may provide records for subjects to the health technology system 110 via, for example, a secure network 150, such that the health technology system 110 can perform various analytics on the records and provide a visualization of the records of one or more subjects. In one instance, the health provider 116 receives, through the interface 120, a visualization of a subject's health and medical history generated by the visualization system of the health technology system 110 in conjunction with or without the learning system. A detailed description of the visualization is described in conjunction with FIGS. 2 through 4.

The health providers 116 and the health technology system 110 are configured to communicate via the network 150, which may comprise any combination of local area and/or wide area networks, using both wired and/or wireless communication systems. In one embodiment, the network 150 uses standard communications technologies and/or protocols. For example, network 150 includes communication links using technologies such as Ethernet, 802.11, worldwide inter-operability for microwave access (WiMAX), 3G, 4G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In one embodiment, all or some of the communication links of the network 150 may be encrypted using any suitable technique or techniques.

FIG. 2 illustrates a block architecture of the health technology system 110, according to one embodiment. In one embodiment, the health technology system 110 includes a visualization system 210 and a learning system 225. The health technology system 110 also includes a records datastore 280 and a model datastore 285. In other embodiments, the health technology system 110 may include additional, fewer, or different components for various types of applications. Conventional components such as network interfaces, security functions, load balancers, failover servers, management and network operations consoles, and the like are not shown so as to not obscure the details of the system architecture.

The records datastore 280 includes various types of health and medical records for one or more subjects that are obtained from, for example, different health providers 116, such as hospitals, medical clinics and facilities, laboratories, pharmacies, and the like. For example, the records datastore 280 may include hospital visit records indicating a visit time of a patient, any diagnoses decided on the visit, prescriptions by the physician, and the like. As another example, the records datastore 280 may also include prescription records obtained from pharmacies. In one embodiment, because the records stored in the records datastore 280 contain private and sensitive medical information, the health technology system 110 may configure the records store 280 with various security and access control measures.

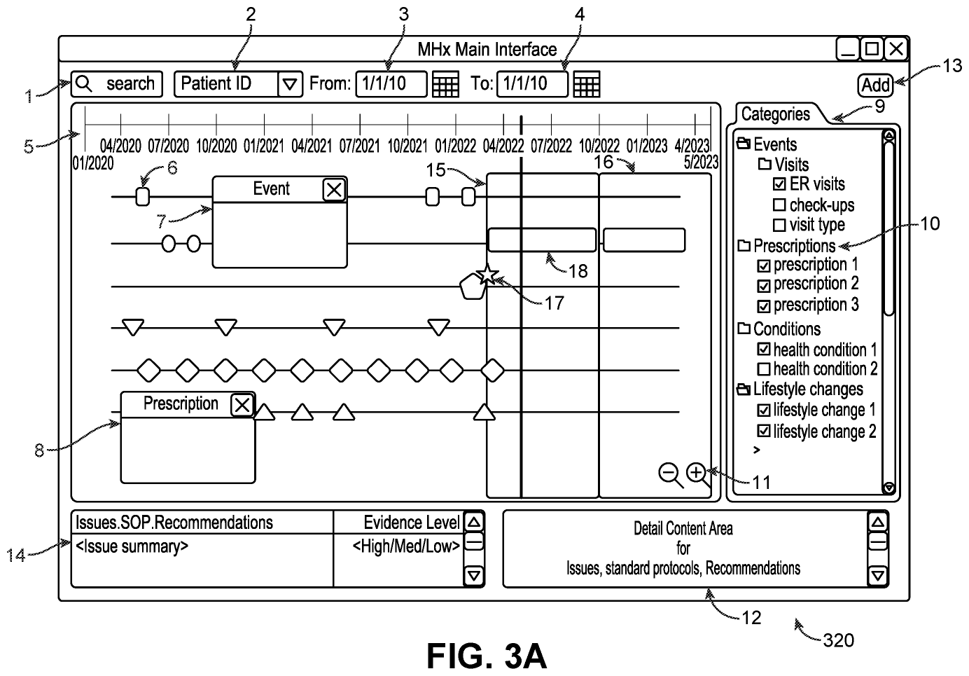

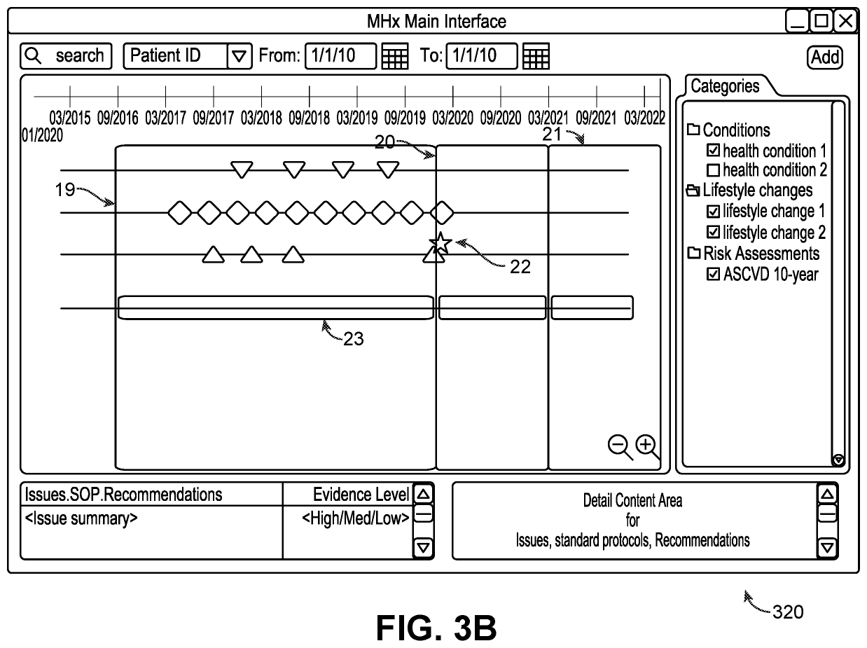

The visualization system 210 generates an interface on computing systems of health providers 116 (or any other computing system) that displays health and medical history of one or more subjects based on a set of input categories. For a particular subject, the visualization system 210 obtains relevant records from the record datastore 280 and generates an interface 120 on the computing system. A more detailed description of example interfaces is provided below in conjunction with FIGS. 3A through 5.

FIG. 3A illustrates an interface 320 generated by the visualization system 210, according to one embodiment. As shown in FIG. 3A, the interface 320 may display graphics components for displaying the medical history of one or more subjects or predictions generated for the subjects. In one embodiment, the interface 320 allows the user or operator of the interface 320 to search for existing patients via unique identifiers or keywords such as medical record numbers (MRN), first name, or last name of the patient. Results of such a query are displayed in the dropdown element (e.g., element “2” in FIG. 3A) for the operator to display all the medical history of the selected patient in the interface 320.

In one embodiment, the interface 320 may also include date field elements (e.g., elements “3” and “4” in FIG. 3A) that can be set using the calendar widget (popup) to select the viewing date range. These fields are initially set to blank, and when a particular patient is selected (when the date fields are blank) then the date fields may be populated with the earliest and the latest date of the patient's medical history. Whenever a patient is selected using the dropdown element 2, these date field elements update to the newly selected patient's medical history dates (earliest and latest) accordingly.

Upon selection of the start and the end date to display, the main panel window updates with the patient's medical records. In one embodiment, the events for the subject are grouped according to different input categories, such as record type (e.g., hospital visits, laboratory tests, procedures) and displayed on separate timelines, for example, for each input category. An input category may refer to a type of health or medical related event for the subject, and may include, but is not limited to, types of visits (e.g., emergency room visits, in-patient or out-patient visits), laboratory tests, medical procedures, diagnoses, prescriptions for a particular drug, onset of health conditions, significant lifestyle changes, any other events captured in the medical records of the subject.

For example, in the example interface 320 shown in FIG. 3A, the example input categories include ER visits, prescription events of drug 1 (“prescription 1”), prescription events of drug 2 (“prescription 2”), prescription events of drug 3 (“prescription 3”), a first health condition (“health condition 1”), and lifestyle changes, including a first lifestyle change (“lifestyle change 1”) and a second lifestyle change (“lifestyle change 2”). The visualization system may identify and assign each event in the history to a relevant input category and display the event at a corresponding time on the timeline for that input category. For example, the first timeline indicates an ER visit event with the visual indicator (element “6” in FIG. 3A) that corresponds to a time the subject made an ER visit. The horizontal axis may correspond to an amount of time (e.g., year of time, five years of time), and the visual indicators may be presented at a corresponding time point on the timeline. Similarly, the remaining timelines indicate a corresponding type of event with visual indicators shaped as circles for the second timeline, a pentagon for the third timeline, an inverse triangle for the fourth timeline, a diamond for the fifth timeline, and a triangle for the sixth timeline.

In one instance, the interface 320 includes one or more interactive elements. As an example, hovering over or clicking on the icon which represents one event within a respective timeline result in a display of a popup window element (e.g., element “7” and element “8” in FIG. 3A) showing the full name, the date, and the type and other relevant information of the event.

In one embodiment, a panel on the right (or any) side of the interface may show the full list of display items (i.e., input categories) that can be displayed for the selected patient. This list does not show those event types that are not present in the patient's medical history. For example, if the patient does not have any ER visit records, no selection box would be displayed in the list. By default, all record types in the list are selected to display, but this default behavior can be configured in the user's preferences store. By checking and unchecking each selection box, the operator can control which type of records are displayed in the main panel.

In one embodiment, the main timeline panel content can be zoomed in and out using the magnifying glass icons (e.g., element “11” in FIG. 3A). With click-hold-drag, a particular time window can be selected. When a time window is selected, the start and the end date field (e.g., elements “3” and “4” in FIG. 3A) are updated accordingly. When displaying timelines that extend the viewable area of the window, the content can be shifted left (to display older timeline content), or right (to display newer timeline content), up, or down.

In one embodiment, in addition to displaying existing medical records of a particular patient, the interface 320 can display predictive medical outcomes or incident probabilities within the same interface 320. As described above in conjunction with the system environment 100 of FIG. 1, the visualization system 210 may be in communication with a learning system 225 within the health technology system 110 that trains models and generates predictions for the patient using one or more machine-learning or artificial intelligence models. For example, the visualization system 210 may obtain likelihoods of potential adverse drug events or side effects. The visualization system 210 receives predictions generated by the learning system 225 and displays prediction information on the interface 320, such that predictive risk assessments can be informed to a user based on a subject's health and medical history.

In one embodiment, the visualization system 210 highlights sections (e.g., element “15” in FIG. 3A) of the timelines to show a relevant time period of such prediction information and/or probability of future events. For example, the container (e.g., element “15” in FIG. 3A) may represent a period of time where it is 85% likely to develop a bleeding episode based on the patient's health and medical history including laboratory results, diagnoses, procedures, prescriptions, and other clinical factors. A lower probability of incidents may be displayed with a container with a different color or hash or any other method to visualize a difference (e.g., element “16” in FIG. 3A). The details of such outcome prediction displays when the operator clicks or hovers over an element (e.g., the box element “18” in FIG. 3A) which covers the entire time period.

In one embodiment, the visualization system 210 may be in communication with an analytics system 230 that provides information on potential issues, standard protocols and deviations from these standard protocols, recommendations, and the like, based on the health and medical history of a subject. For example, the visualization system 210 can display expected standard protocols identified based on rule-based lookup of recent medical records by the analytics system 230. Moreover, the timeline of events for the patient may show a series of prescriptions and procedures that do not follow standard protocols. The visualization system 210 in conjunction with the learning system 225 or the analytics system 230 may display an indication flagging this deviation on the interface 320.

In one embodiment, the visualization system 210 also receives, from the analytics system 230, various risk assessment scores from one or more risk estimators that are, for example, widely recognized by the health and medical industry, such as the Atherosclerotic Cardiovascular Disease (ASCVD) Risk Estimator Plus from the American College of Cardiology (ACC).

FIG. 3B illustrates a continuation of the main interface generated by the visualization system, according to one embodiment. In the example interface of FIG. 3B, the visualization system 210 receives ASCVD risk estimates from a ASCVD Risk Estimator Plus from ACC. The risk assessment scores may include a 10-year ASCVD risk and/or a lifetime ASCVD risk. Responsive to toggling the “ASCVD 10-year risk” selection on the right panel, the interface 320 may also show one or more containers that represent relevant periods of time of when the risk assessment scores are relevant.

As an example, the subject for the visualization interface 320 may be of 57 years of age at May 2023. The risk assessment score for 10-year ASCVD risk at 50 years of age was only 5.9%, which is borderline risk. However, the subject may have started smoking at 55 years of age, which is a significant lifestyle event. At that point, the 10-year ASCVD risk for the subject may be updated to 12.6% and lifetime ASCVD risk may be 50%, which is an intermediate level of risk. At 57 years of age, the subject may have started hypertension treatment, which further increased the risk. The subject is now at 10-year ASCVD risk of 20.1% and lifetime ASCVD risk of 69%, which is a high level of risk.

Therefore, one container (e.g., element “19” in FIG. 3B) may indicate the borderline level of 10-year ASCVD risk from when the subject was 50 years of age to 55 years of age, another container (e.g., element “20” in FIG. 3B) may indicate the intermediate level of 10-year ASCVD risk from when the subject was 55 years to 57 years of age, and yet another container (e.g., element “21” in FIG. 3B) may indicate a high level of 10-year ASCVD risk from when the subject was 57 years of age to present. In particular, starting of smoking for the subject may be indicated by a visual indicator (e.g., element “22” in FIG. 3B) on a lifestyle change timeline, and starting of hypertension treatment for the subject may also be indicated by a visual indicator for another timeline.

Returning to FIG. 3A, in one embodiment, the interface 320 may show the various types of information obtained from the learning system 225 and/or the analytics system 230 in a portion such as a panel (e.g., element “14” in FIG. 3A) on the interface 320. Specifically, the panel 14 may display a list of items for consideration of the user based on the intelligence provided by the learning system 225, the analytics system 230, and other modules of the heath technology system 110.

Specifically, the panel may display an item that refers to potential issues determined from the trained machine-learning models generated by the learning system 225, such as high likelihood of adverse drug reaction (ADR) due to a certain prescription or other related incidents. As another example, the panel may display standard medical protocol computed based on the latest diagnoses or lab results of the subject. As another example, the panel may display prescription recommendations for the subject. For example, if genotype data of the subject is available, the prescription dosage recommendations from sources such as Clinical Pharmacogenetics Implementation Consortium (CPIC) or Food and Drug Administration (FDA) may be shown. As another example, the panel may display prescription dosage guidelines determined based on the prescription records of the subject. As another example, the panel may display applicable black box warnings based on the current set of prescribed medications as well as any drug-drug interactions, drug-food interactions, use in specific populations using patient data (pregnancy, pediatric, geriatric, renal impairment) or contraindications.

In one embodiment, when an item is selected from the panel 14 of the interface 320, the interface 320 may also include an element (e.g., element “12” in FIG. 3A) that shows details that may be associated with the selected issue. At the same time, the events of the subject that are relevant to the selected item, such as related prescription events, laboratory tests, and/or diagnoses events are marked with an indicator (e.g., star-shaped element “17” in FIG. 3A). Zero or more items may be indicated in this manner. For example, the third timeline may indicate events (pentagon shaped indicator) for a particular prescription drug. An item for display in the panel 14 may be high likelihood of an adverse drug reaction due to drug-drug interactions between the prescription drug and another drug that is being consumed by the patient. Thus, responsive to selection of that item in the panel 14, details of the identified drug-drug interaction may be described in element 12, and the relevant event may be marked with the star-shaped indicator.

Returning to FIG. 2, the learning system 225 obtains information from the records datastore 280 on one or more subjects and applies machine-learning and/or artificial intelligence models to generate predictions. The learning system 225 may provide the predictions and any associated information on the predictions to the visualization system 210, such that the prediction information can be displayed on the interface as described in conjunction with FIGS. 3A-3B. In particular, the learning system 225 can be configured to perform inference tasks on longitudinal health and medical records of subjects (e.g., obtained from timeline of events). Thus, the prediction information generated by the learning system 225 may include predicted likelihoods (e.g., of ADR's or side effects), relevant periods of time applicable to the predicted likelihoods, and the like that are displayed as highlighted sections on the interface, such as elements 15 and 16 on example interface 320.

In one instance, models trained by the learning system 225 may be configured to receive longitudinal health and medical records of the subject and generate one or more predictions for the subject. The longitudinal health and medical records for a subject may be a collection of events that have or are expected to occur for the subject over a predetermined period of time (e.g., events occurred last five years, ten years, twenty years, lifetime). For example, a trained model may receive a collection of events that have occurred for the patient in the last five years and predict an 85% likelihood that the patient will experience a bleeding episode for the relevant time period of December 2022 through March 2023. The relevant time period can be displayed on the interface 120 as a highlighted section.

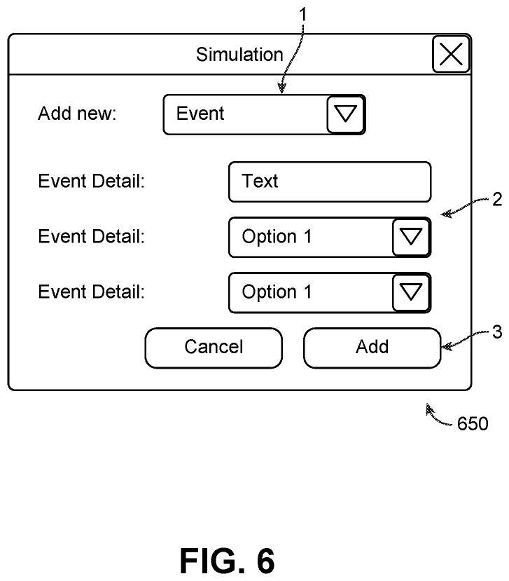

Moreover, as described in more detail below in conjunction with FIG. 6, the learning system 225 is also configured to receive one or more updates to the timelines of a subject, for example, to incorporate one or more simulated events for the subject. The learning system 225 may re-apply trained models to updated longitudinal health and medical records that incorporate the simulated events to generate a set of updated prediction information. The learning system 225 provides the updated prediction information to the visualization system 210 for display on the interface 120.

In one embodiment, the machine-learning models trained and used by the learning system 225 is configured to perform a variety of inference and generative tasks, for example, health event classification, natural language processing (NLP), image processing, video processing, or audio processing applications in the context of healthcare applications. The models are configured as one or a combination of artificial neural networks (ANNs), recurrent neural networks (RNNs), deep learning neural networks (DNNs), bi-directional neural networks, transformers, classifiers such as support vector machines (SVMs), decision trees, regression models such as logistic regression, linear regression, stepwise regression, generative models such as transformer-based architectures including GPT, BERT, encoder-decoders, variational autoencoders (VAE's), diffusion models, cross-modal transformer architectures, and the like. The trained models are stored in the model datastore 285.

The learning system 225 may train the machine-learning models using training data including one or more training instances. A training instance includes training input data and output data. The training output data may include a set of known labels for the corresponding set of training input data. For example, for training a model on ADR prediction, the training input data may include known longitudinal health and medical history obtained for a previous patient from the records datastore 280, and the training output data may include a known ADR that occurred for the patient during a relevant period of time. The learning system 225 may train the models by performing a forward pass to generate estimated outputs by applying the model to the training inputs, computing a loss between the estimated outputs and the known outputs, and backpropagating the loss function to update the parameters. This process is repeated until a convergence criterion is reached.

The analytics system 230 generates information on potential issues, standard protocols and deviation from the protocols, recommendations, and the like based on the health and medical history of the subject and the knowledge bank store 290. Specifically, the knowledge bank store 290 is a datastore of health and medical data, including but not limited to, standard protocols or procedures, genotype data, prescription dosage recommendations from sources such as CPIC and/or FDA, prescription dosage guidelines, black box warnings, drug interaction data such as drug-drug interactions, drug-food interactions, use in specific populations (e.g., pregnancy, pediatric, geriatric, renal impairment), and/or contraindications.

The analytics system 230 obtains longitudinal health and medical records of a subject from the records datastore 280 and generates analytical information in conjunction with the data stored in the knowledge bank store 290. The analytical information may be customized for the particular subject. The analytics system 230 provides the information to the visualization system 210, such that the analytical information can be displayed on the interface as described in conjunction with FIGS. 3A-3B.

For example, the analytics system 230 may obtain a series of prescriptions and medical procedures of a patient, identify one or more relevant standard protocols for the patient, and identify prescriptions and procedures that do not follow the standard protocols. As another example, the analytics system 230 may obtain genotype information for a subject and determine prescription dosage recommendations from sources such as CPIC and/or FDA. As yet another example, the analytics system 230 obtains a list of prescribed medications for a patient and determines black box warnings for the prescribed medications and drug interaction data from the knowledge bank store 290. The analytics system 230 provides the information to the visualization system 210, such that the analytics information is displayed on the interface at, for example, the panel 14 or any other appropriate location.

In one embodiment, the analytics system 230 is connected to one or more risk assessment systems, such risk estimator, that generate risk scores for various health and medical conditions. The analytics system 230 can obtain the longitudinal records for a subject and input the information to generate one or more risk assessment scores using a risk estimator. The analytics system 230 may provide the risk scores to the visualization system 210, such that the scores can be presented on the interface, as described in conjunction with FIGS. 3A-3B.

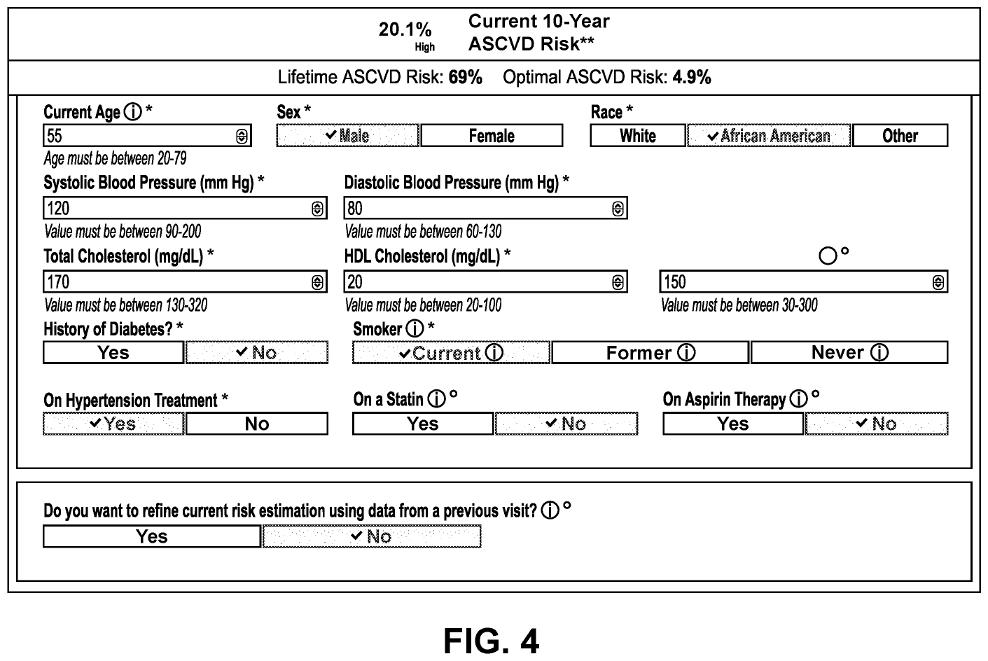

FIG. 4 illustrates an example risk estimator for ASCVD, according to one embodiment. The example ASCVD risk estimator includes data fields for inputting information for a subject, including the current age, sex, race, systolic blood pressure (mm Hg), diastolic blood pressure (mm Hg), total cholesterol (mg/dL), HDL cholesterol (mg/dL), and LDL cholesterol (mg/dL), history of diabetes, whether the subject is a smoker, whether the subject is undergoing hypertension treatment, whether the subject in on a statin, and whether the subject is on aspirin therapy. As indicated on the interface, the subject's 10-year ASCVD risk is estimated as 20.1% and the lifetime risk is estimated as 69%.

Moreover, as described in more detail below in conjunction with FIG. 6, the analytics system 230 is also configured to receive one or more updates to the timelines of a subject, for example, to incorporate one or more simulated events for the subject. The analytics system 230 updates the information to incorporate the simulated events, for example, the information may be updated with new black box warnings or drug interaction data for a newly added prescription event for the drug Metformin. The analytics system 230 provides the updated analytics information to the visualization system 210 for display on the interface 120.

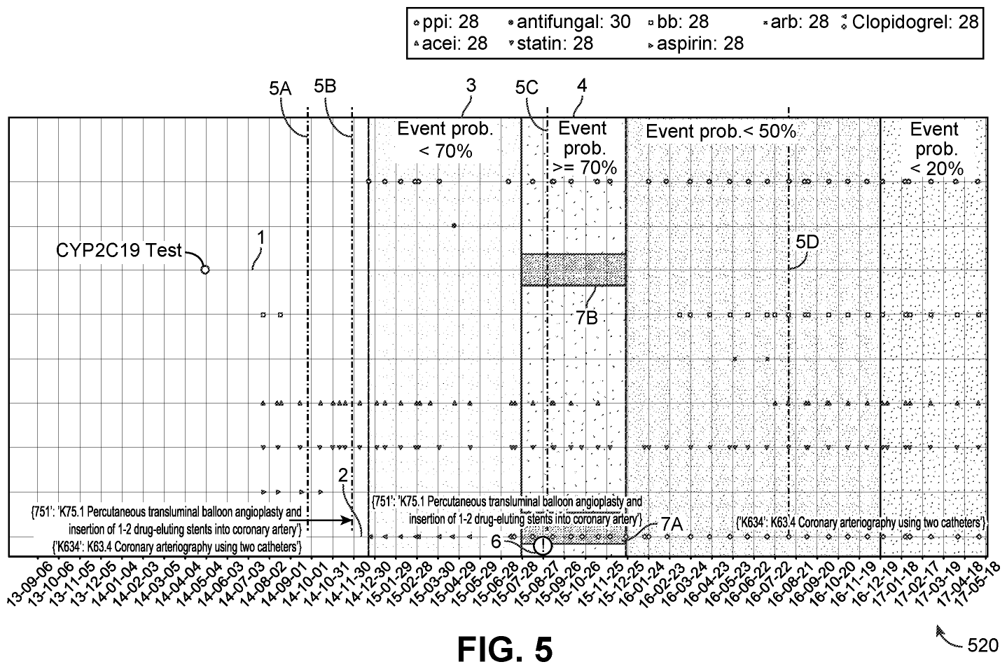

FIG. 5 illustrates a main interface 520 generated by the visualization system 210, according to another embodiment. The example interface 520 illustrates the health and medical history of a patient that made multiple hospital visits and took multiple prescriptions among other events. The timelines illustrated in FIG. 5 each represent different type of prescriptions taken by the patient. For example, the CYP2C19 timeline shown by element “1” in FIG. 5 illustrates when the patient was genotyped for CYP2C19 (indicated by label “CYP2C19 Test”). The clopidogrel prescription timeline shown by element “2” in FIG. 5 illustrates clopidogrel prescription events, including details of how many pills per month were prescribed to the patient. The left edge of the highlighted box (indicated by element “3” in FIG. 5) indicates a time period in which the patient is predicted to experience an ADR event at a probability of 70% or less. The likelihood may be determined based on a holistic, longitudinal view of the patient's records. The box (indicated by element “4” in FIG. 5) indicates a time period in which the probability of adverse drug reaction events is equal to or greater than 70%.

The dashed lines (indicated by elements “5A,” “5B,” “5C,” “5D” in FIG. 5) indicate times that the patient made hospital visits. These may include ER visits or visits for medical procedures. Each dashed line is associated with a caption that indicates what type of event occurred. The indication shown by the exclamation mark (indicated by element “6” in FIG. 5) on the clopidogrel timeline indicates an ADR event and the two red bands (indicated by elements “7A” and “7B” in FIG. 5) indicate the events (CYP2C19 test and clopidogrel prescriptions) that are relevant to the ADR event.

### Simulation of Future Events in Health Technology System

FIG. 6 illustrates a dialog box 650 for simulating a new event for subjects, according to one embodiment. In one embodiment, the dialog box 650 may be generated when the user interacts with the “add” button (element “13” in FIG. 3A) shown in the example interface 320. In one embodiment, the health technology system 110 allows a user to simulate one or more future, present, or past events for a subject. Thus, by simulating an event, a user of the health technology system 110 allows a user to simulate different types of events and scenarios, such as potential prescription events for a drug, diagnoses, medical procedures, and the like. The components of the health technology system 110 can process and analyze the simulated events to determine whether there are any potential issues, ADR's, applicable black box warnings, or drug dosage recommendations for the subject.

In one instance, a user of the health technology system 110 can select through a graphics element (e.g., element “1” in FIG. 6) what type of event (e.g., ER visit, prescription, test result, etc.) the user would like to add to the existing display of the timelines. Based on the selected event type in the dropdown element, event detail prompts (e.g., elements “2” in FIG. 6) may be displayed below. The prompts may ask for information such as prescription dosage amount and frequency when adding a new prescription event. As another example, a diagnosis code may be requested for a diagnosis event.

In one embodiment, the user completes the process of adding a simulated event by pressing an add button (e.g., element “3” in FIG. 6). This action may close the dialog box 550 and allow the user to place the new event on any of the existing timelines in the generated interface 120. For example, a user may add a potential sclerotherapy procedure on a timeline for a patient on a future date of May 7, 2023, to determine how the simulated event would affect any risk assessment predictions or issues to consider for the patient.

Upon placing the new event on a timeline, details of the simulated event may be provided to components of the health technology system 110, such as the learning system 225 or the analytics system 230. The longitudinal health and medical records of the subject is updated to incorporate the newly added event, and the learning system 225 may generate updated prediction information reflecting the simulated events of the subject. As another example, the analytics system 230 may generate updated analytics information reflecting the simulated events of the subject. The visualization system 210 may receive the updated prediction or analytics information and update the interface 120 to reflect the new information. For example, the panel (e.g., element “14” in FIG. 3A) is updated based on the simulated events. The user may also remove the added event by pressing the delete button while selected.

For example, in the example interface 320 of FIG. 3A, the pentagon-shaped element may indicate presence of a simulated event that corresponds to a patient consuming a particular prescription drug in the future. The learning system 225 may obtain updated longitudinal records for the subject and generate ADR predictions that indicate whether any adverse responses will be triggered for the drug. The visualization system 210 receives the updated prediction information and displays a star-shaped element “17” indicating a significant likelihood of ADR. The visualization system 210 highlights a section (e.g., element “15” in FIG. 3A) corresponding a time period relevant to the ADR prediction and indicating at least an 85% likelihood of triggering an ADR.

As another example, a clinician may simulate different events and how that affects the risk assessment scores, via the example dialog box 550. For example, for the example ASCVD risk estimator, the clinician may input event updates for a subject, such as changes in LDL cholesterol or HDL cholesterol levels, change in lifestyle changes (e.g., subject stops smoking), to simulate how these changes update a subject's ASCVD risk assessment score. The updated risk assessment scores can be visualized on the interface along with simulated events for the subject.

In this manner, the health technology system 110 allows users to flexibly simulate various types of health and medical related events using the interface 120 generated by the visualization system 210. Moreover, because the visualization system 210 works seamlessly with other components such as the learning system 225 and the analytics system 230, the health and medical related information can be quickly updated such that the user can efficiently be aware of any issues that can occur if the simulated events were to happen for the subject.

### Method of Incorporating Simulated Events in Visualization Interface

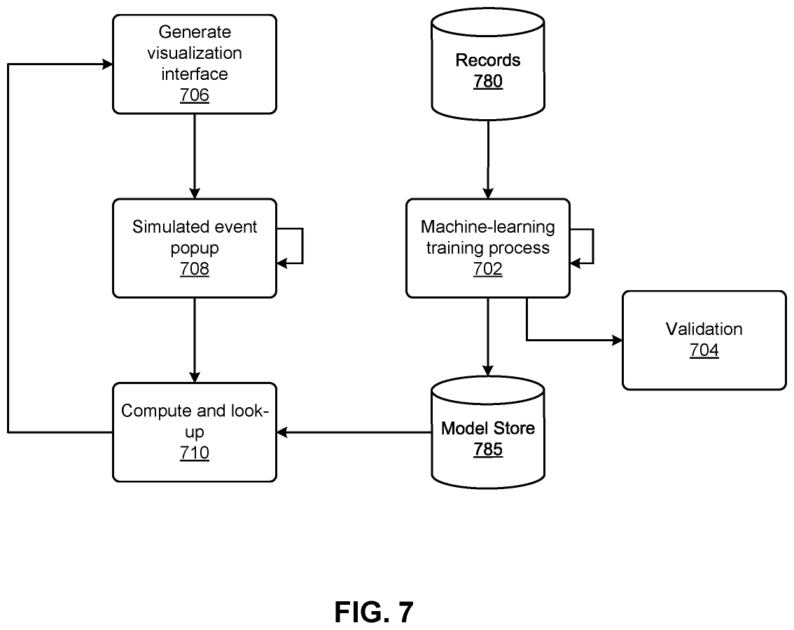

FIG. 7 illustrates a method of using machine-learning models trained using longitudinal medical records and obtaining the results by the visualization system 210, according to an embodiment. One or more processes illustrated in FIG. 7 may be performed by the various components of the health technology system 110, including the visualization system 210, the learning system 225, and/or the analytics system 230.

As shown in FIG. 7, health and medical records of subjects, including longitudinal health and medical records, may be used in a machine-learning training process. In one instance, the longitudinal records may be obtained from a records data store 780 configured substantially similar or identical to the records datastore 280 described in conjunction with FIG. 2. The longitudinal records are used to train 702 one or more machine-learning models by the learning system 225. The training process may occur based on genomic and/or clinical risk factors of the subjects. The parameters of the prediction models are optimized through a backpropagation process. The parameters of the ML models are stored in a model datastore 785 configured substantially similar or identical to the model datastore 285 described in conjunction with FIG. 2.

The visualization system 210 may generate 706 the visualization interface 120 described herein and to display timelines indicating health and medical events for a subject. In one instance, the longitudinal health and medical records of a subject are used to generate prediction information, such as ADR predictions, using the trained models, and used to generate analytics information including drug dosage recommendations, black label warnings, standard protocols and deviations from the protocols for the subject. The prediction information and analytics information may be provided to the visualization system 210, so that the visualization system 210 can overlay the information on the interface 120 in association with timelines for the subject.

At a subsequent time, a new event may be simulated 708 via a popup element on the interface 120. The popup element may be a dialogue element in which details for the simulated event is input, and the new event may be confirmed by the visualization system 210. The longitudinal records of the subject are updated to incorporate the newly added events. After, the learning system 225 and the analytics system 230 may perform a compute and look-up 710 to generate updated prediction information and analytics information to reflect the newly simulated events. The visualization system may refresh the patient's timelines and information panel (e.g., element “4” in FIG. 3A) according to incorporate the newly simulated events.

### Method for Displaying Health and Medical Records

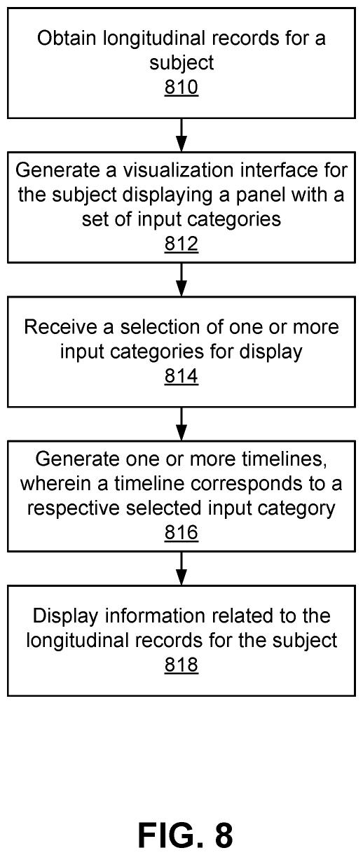

FIG. 8 is an example flowchart for displaying health and medical records for a subject, according to one embodiment. The health technology system 110 obtains 810 longitudinal records for a subject. In one embodiment, the longitudinal records describe one or more health and medical events for the subject. The health technology system 110 generates 812, on a client device, a visualization interface for the subject, the visualization interface displaying at least a panel including a set of input categories. An input category may correspond to a respective type of health and medical event. The health technology system 110 receives 814, from a user of the client device, a selection of one or more input categories for display in the visualization interface.

The health technology system 110 generates 816, on the client device, one or more timelines on the visualization interface. In one embodiment, a timeline corresponds to a respective selected input category. The timeline may include one or more sequentially placed visual indicators. In one embodiment, a visual indicator of a timeline corresponds to occurrence or expected occurrence of an event of the selected input category type at a corresponding time point on the timeline. The health technology system 110 displays 818 information related to the longitudinal records for the subject on the visualization interface.

### SUMMARY

The foregoing description of the embodiments of the disclosed configuration has been presented for the purpose of illustration; it is not intended to be exhaustive or to limit the disclosed configuration to the precise forms disclosed. Persons skilled in the relevant art can appreciate that many modifications and variations are possible in light of the above disclosure.

Some portions of this description describe the embodiments of the disclosed configuration in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the disclosed configuration may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the disclosed configuration may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the disclosed configuration be limited not by this detailed description, but rather by any claims that issue on an application based hereon. Accordingly, the disclosure of the embodiments of the disclosed configuration is intended to be illustrative, but not limiting, of the scope of the disclosed configuration, which is set forth in the following claims.

What is claimed is:

1. A method, comprising:
obtaining longitudinal records for a subject, the longitudinal records describing one or more health and medical events for the subject;
generating, on a client device, a visualization interface for the subject, the visualization interface displaying at least a panel including a set of input categories, wherein an input category corresponds to a respective type of health and medical event;
receiving, from a user of the client device, a selection of one or more input categories for display in the visualization interface;
generating, on the client device, one or more timelines on the visualization interface, wherein a timeline corresponds to a respective selected input category, and wherein the timeline includes one or more sequentially placed visual indicators, wherein a visual indicator of a timeline corresponds to occurrence or expected occurrence of an event of the selected input category type at a corresponding time point on the timeline; and
displaying information related to the longitudinal records for the subject on the visualization interface.

2. The method of claim 1, further comprising:
generating prediction information including one or more predictions for the subject by applying a trained machine-learned model to at least a portion of the longitudinal records for the subject; and
displaying a highlighted section on the visualization interface that corresponds to a time period the prediction information is relevant for the subject.

3. The method of claim 2, wherein the one or more predictions are predictions for an adverse drug reaction (ADR).

4. The method of claim 2, wherein the one or more predictions includes a first prediction of a first likelihood and a second prediction of a second likelihood, and wherein the highlighted section corresponds to the time period relevant to the first prediction, and the method further comprising:
displaying a second highlighted section on the visualization interface that corresponds to a second time period relevant to the second prediction.

5. The method of claim 1, further comprising:
receiving, from the user of the client device, an indication to simulate a set of events for the subject;
updating the longitudinal records for the subject to incorporate the set of simulated events; and
generating one or more predictions for the subject by applying a trained machine-learned model to at least a portion of the updated longitudinal records for the subject.

6. The method of claim 5, further comprising:
responsive to receiving the indication, prompting a dialogue box configured to receive details of the set of simulated events from the user.

7. The method of claim 1, further comprising:
identifying analytics information including one or a combination of standard protocols relevant to the subject, deviations from the standard protocols, and prescription dosage recommendations from the longitudinal records of the subject; and
displaying the analytics information on the visualization interface.

8. A non-transitory computer readable medium comprising stored instructions, the stored instructions when executed by at least one processor of one or more computing devices, cause the one or more computing devices to:
obtain longitudinal records for a subject, the longitudinal records describing one or more health and medical events for the subject;
generate, on a client device, a visualization interface for the subject, the visualization interface displaying at least a panel including a set of input categories, wherein an input category corresponds to a respective type of health and medical event;
receive, from a user of the client device, a selection of one or more input categories for display in the visualization interface;
generate, on the client device, one or more timelines on the visualization interface, wherein a timeline corresponds to a respective selected input category, and wherein the timeline includes one or more sequentially placed visual indicators, wherein a visual indicator of a timeline corresponds to occurrence or expected occurrence of an event of the selected input category type at a corresponding time point on the timeline; and
display information related to the longitudinal records for the subject on the visualization interface.

9. The non-transitory computer readable medium of claim 8, the instructions further causing the one or more computing devices to:
generate prediction information including one or more predictions for the subject by applying a trained machine-learned model to at least a portion of the longitudinal records for the subject; and
display a highlighted section on the visualization interface that corresponds to a time period the prediction information is relevant for the subject.

10. The non-transitory computer readable medium of claim 9, wherein the one or more predictions are predictions for an adverse drug reaction (ADR).

11. The non-transitory computer readable medium of claim 9, wherein the one or more predictions includes a first prediction of a first likelihood and a second prediction of a second likelihood, and wherein the highlighted section corresponds to the time period relevant to the first prediction, and the instructions further causing the one or more computing devices to:
display a second highlighted section on the visualization interface that corresponds to a second time period relevant to the second prediction.

12. The non-transitory computer readable medium of claim 8, the instructions further causing the one or more computing devices to:
receive, from the user of the client device, an indication to simulate a set of events for the subject;
update the longitudinal records for the subject to incorporate the set of simulated events; and
generate one or more predictions for the subject by applying a trained machine-learned model to at least a portion of the updated longitudinal records for the subject.

13. The non-transitory computer readable medium of claim 12, the instructions further causing the one or more computing devices to:
responsive to receiving the indication, prompt a dialogue box configured to receive details of the set of simulated events from the user.

14. The non-transitory computer readable medium of claim 8, the instructions further causing the one or more computing devices to:
identify analytics information including one or a combination of standard protocols relevant to the subject, deviations from the standard protocols, and prescription dosage recommendations from the longitudinal records of the subject; and
display the analytics information on the visualization interface.

15. A computer system comprising:
one or more computer processors; and
one or more computer readable mediums storing instructions that, when executed by the one or more computer processors, cause the computer system to:
obtain longitudinal records for a subject, the longitudinal records describing one or more health and medical events for the subject;
generate, on a client device, a visualization interface for the subject, the visualization interface displaying at least a panel including a set of input categories, wherein an input category corresponds to a respective type of health and medical event;
receive, from a user of the client device, a selection of one or more input categories for display in the visualization interface;
generate, on the client device, one or more timelines on the visualization interface, wherein a timeline corresponds to a respective selected input category, and wherein the timeline includes one or more sequentially placed visual indicators, wherein a visual indicator of a timeline corresponds to occurrence or expected occurrence of an event of the selected input category type at a corresponding time point on the timeline; and
display information related to the longitudinal records for the subject on the visualization interface.

16. The computer system of claim 15, the instructions further causing the computer system to:
generate prediction information including one or more predictions for the subject by applying a trained machine-learned model to at least a portion of the longitudinal records for the subject; and
display a highlighted section on the visualization interface that corresponds to a time period the prediction information is relevant for the subject.

17. The computer system of claim 16, wherein the one or more predictions are predictions for an adverse drug reaction (ADR).

18. The computer system of claim 16, wherein the one or more predictions includes a first prediction of a first likelihood and a second prediction of a second likelihood, and wherein the highlighted section corresponds to the time period relevant to the first prediction, and the instructions further causing the one or more computer system to:
display a second highlighted section on the visualization interface that corresponds to a second time period relevant to the second prediction.

19. The computer system of claim 15, the instructions further causing the computer system to:
receive, from the user of the client device, an indication to simulate a set of events for the subject;
update the longitudinal records for the subject to incorporate the set of simulated events; and
generate one or more predictions for the subject by applying a trained machine-learned model to at least a portion of the updated longitudinal records for the subject.

20. The computer system of claim 19, the instructions further causing the computer system to:
responsive to receiving the indication, prompt a dialogue box configured to receive details of the set of simulated events from the user.
